(19) United States
(12) Patent Application Publication
(10) Pub. No.: US 2004/0039017 A1
(43) Pub. Date: Feb. 26, 2004

(54) Urotensin-II receptor antagonists

(76) Inventors: Dashyant Dhanak, King of Prussia, PA (US); Steven d. Knight, King of Prussia, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/450,106

(22) PCT Filed: Dec. 5, 2001
(86) PCT No.: PCT/US01/47451

(30) Foreign Application Priority Data
Dec. 11, 2000 (US) 60254600

(51) Int. Cl. A61K 31/4709; A61K 31/47

(52) U.S. Cl. 514/312

(57) ABSTRACT

The present invention relates to a method of antagonizing the Urotensin-II receptor by 2-(NH-substituted) quinolones.

### FIELD OF THE INVENTION

The present invention relates generally to a method of antagonizing the Urotensin-II receptor by 2-(NH-substituted) quinolones.

### BACKGROUND OF THE INVENTION

The integrated control of cardiovascular homeostasis is achieved through a combination of both direct neuronal control and systemic neurohormonal activation. Although the resultant release of both contractile and relaxant factors is normally under stringent regulation, an aberration in this status quo can result in cardiohemodynamic dysfunction with pathological consequences.

The principal mammalian vasoactive factors that comprise this neurohumoral axis, namely angiotensim-II, endothelin-1, norepinephrine, all function via an interaction with specific G-protein coupled receptors (GPCR). Urotensin-II, represents a novel member of this neurohumoral axis.

In the fish, this peptide has significant hemodynamic and endocrine actions in diverse end-organ systems and tissues:

smooth muscle contraction

both vascular and non-vascular in origin including smooth muscle preparations from the gastrointestinal tract and genitourinary tract. Both pressor and depressor activity has been described upon systemic administration of exogenous peptide

osmoregulation:

effects which include the modulation of transepithelial ion (Na⁺, Cl⁻) transport. Although a diuretic effect has been described, such an effect is postulated to be secondary to direct renovascular effects (elevated GFR)

metabolism:

urotensin-II influences prolactin secretion and exhibits a lipolytic effect in fish (activating triacylglycerol lipase resulting in the mobilization of non-esterified free fatty acids)

(Pearson, et. al. Proc. Natl. Acad. Sci. (U.S.A.) 1980, 77, 5021; Conlon, et. al. J. Exp. Zool. 1996, 275, 226.)

In studies with human Urotensin-II it was found that it:

was an extremely potent and efficacious vasoconstrictor

exhibited sustained contractile activity that was extremely resistant to wash out

had detrimental effects on cardiac performance (myocardial contractility)

Human Urotensin-II was assessed for contractile activity in the rat-isolated aorta and was shown to be the most potent contractile agonist identified to date. Based on the in vitro pharmacology and in vivo hemodynamic profile of human Urotensin-II it plays a pathological role in cardiovascular diseases characterized by excessive or abnormal vasoconstriction and myocardial dysfunction. (Ames et. al. Nature 1999, 401, 282)

Compounds that antagonize the Urotensin-II receptor may be useful in the treatment of congestive heart failure, stroke, ischemic heart disease (angina, myocardial ischemia), cardiac arrhythmia, hypertension (essential and pulmonary), COPD, restenosis, asthma, (Hay D W P, Luttmann M A, Douglas S A: 2000, Br J Pharmacol: In press.) neurogenic inflammation and metabolic vasculopathies all of which are characterized by abnormal vasoconstriction and/or myocardial dysfunction. Since U-II and GPR14 are both expressed within the mammalian CNS (Ames et. al. Nature 1999, 401, 282), they also may be useful in the treatment of addiction, schizophrenia, impulsivity, anxiety, stress, depression, and neuromuscular function. Functional U-II receptors are expressed in rhabdomyosarcomas cell lines and therefore may have oncological indications. Urotensin may also be implicated in various metabolic diseases such as diabetes (Ames et. al. Nature 1999, 401, 282, Nothacker et al., Nature Cell Biology 1: 383-385, 1999)

### SUMMARY OF THE INVENTION

In one aspect this invention provides for the use of 2(NH-substituted) quinolones for antagonizing the Urotensin-II receptor and thereby treating conditions associated with Urotensin-II imbalance.

In a second aspect, this invention provides for the use of 2(NH-substituted) quinolones for the treatment of congestive heart failure, stroke, ischemic heart disease (angina, myocardial ischemia), cardiac arrhythmia, hypertension (essential and pulmonary), COPD, restenosis, asthma, neurogenic inflammation and metabolic vasculopathies, addiction, schizophrenia, impulsivity, anxiety, stress, depression, neuromuscular function, and diabetes.

The urotensin antagonist may be administered alone or in conjunction with one or more other therapeutic agents, said agents being selected from the group consisting of endothelin receptor antagonists, angiotensin converting enzyme (ACE) inhibitors, vasopeptidase inhibitors, diuretics, digoxin, and dual non-selective β-adrenoceptor and α₁-adrenoceptor antagonists.

Other aspects and advantages of the present invention are described further in the following detailed description of the preferred embodiments thereof.

### DETAILED DESCRIPTION OF THE INVENTION

The present invention provides for a method of treating conditions associated with Urotensin-II imbalance by antagonizing the Urotensin-II receptor which comprises administering to a patient in need thereof, a compound of Formula I:

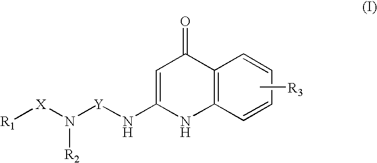

wherein:

R₁ is phenyl, thienyl, benzothienyl, benzhydryl, xanthenyl, napthyl, or indolyl, all of which may be substituted or unsubstituted by one or two substituients selected from: halogens, —CN, CH₃CO—, (C₁₋₆)alkyl, mono to perfluoro(C₁₋₃)alkyl, (C₂₋₆)alkenyl, (C₁₋₆)alkoxy, (C₅₋₁₀)aryloxy, phenylC₍₁₋₆₎alkoxy, —OH, —NH₂, mono- or di-(C₁₋₆)alkylamino, —NO₂, —CO₂H, —CO₂(C₁₋₆)alkyl, —S(C₁₋₆)alkyl, —SO₂(C₁₋₆)alkyl, H₂NSO₂—, —CONH₂, —SO₂(C₅₋₁₀)aryl, or —CO₂N{(C₁₋₆)alkyl}₂;

R₂ is hydrogen or Me;

R₃ is hydrogen, I, F, Br, Cl, C₁₋₆alkyl, C₁₋₆alkoxy, —OH, or —CN;

X is —CH(R₄)— or CO;

R₄ is hydrogen, CO, C₁₋₆ alkyl, or phenyl;

Y is —CH₂C(R₅)(R₆)CH₂—;

R₅ and R₆ are independently hydrogen or C₁₋₆ alkyl;

or a pharmaceutically acceptable salt thereof.

Methods for preparing compounds of Formula I can be found in co-pending PCT application PCT/EP99/02648 which is incorporated by reference herein.

When used herein, the term “alkyl” and similar terms such as “alkoxy” includes all straight chain and branched isomers. Representative examples thereof include methyl, ethyl, n-propyl, iso-propyl, n-butyl, sec-butyl, iso-butyl, t-butyl, n-pentyl and n-hexyl.

When used herein, the terms ’halogen’ and ’halo’ include fluorine, chlorine, bromine and iodine and fluoro, chloro, bromo and iodo, respectively.

By the term “aryl” as used herein, unless otherwise defined, is meant cyclic or polycyclic aromatic C₅-C₁₂ring. Examples are phenyl and naphthyl.

The compounds of the present invention may contain one or more asymmetric carbon atoms and may exist in racemic and optically active form. All of these compounds and their diastereoisomers are contemplated to be within the scope of the present invention.

Preferably R1 is substituted phenyl, thienyl, benzothienyl, benzhydryl, or indolyl. More preferably R₁ is 1-benzyl-3-indolyl, 4,6-dichloro-2-indolyl, 3-trifluoromethylthiophenyl, 2-fluoro-5-trifluoromethylphenyl, 4,6-dichloro-3-methyl-2-indolyl, 6-methoxy-4-trifluoromethyl-2-indolyl, or 3,4-dichlorophenyl, 3,5-dibromophenyl or 4-chloro-3-trifluoromethylbenzyl.

Preferably R₂ is hydrogen;

Preferably R₃ is hydrogen, halo, or alkyl; more preferably R₃ is hydrogen, Cl, or Me.

Preferably X is CH₂.

Preferably Y is CH₂CR₅R₆CH₂, where R₅, and R6 are hydrogen, dimethyl, or forms a cyclic C₍₅₋₆₎ tether; or R₅ is hydrogen; and R₆ is n-propyl, phenyl, benzyl, or methyl. More preferably Y is CH₂CR₅R₆CH₂, wherein R₅, and R6 are H; or R₅ is hydrogen and R₆ is n-propyl, or phenyl.

Preferred Compounds are:

2-{2-[(1-Benzyl-3-indolylmethylamino)methyl]pent-1-ylamino]-1H-quinolin-4-one;

2-{2-[(3-Trifluoromethylthiobenzylamino)methyl]pent-1-ylamino]-1H-quinolin-4-one;

2-{2-[(2-Fluoro-5-trifluoromethylbenzylamino)methyl]pent-1-ylamino]-1H-quinolin-4-one;

2-{2-[(4-n-Butylbenzylamino)methyl]pent-1-ylamino]-1H-quinolin-4-one;

2-{2-[(2,5-Dimethyl-1-phenyl-3-pyrrolylmethylamino)methyl]pent-1-ylamino]-1H-quinolin-4-one;

2-{2-[(1-Bromo-2-naphthylmethylamino)methyl]pent-1-ylamino]-1H-quinolin-4-one;

2-{2-[(4-Bromo-3,5-dimethoxybenzylamino)methyl]pent-1-ylamino]-1H-quinolin-4-one;

2-{2-[(5-Bromo-2-fluorobenzylamino)methyl]pent-1-ylamino]-1H-quinolin-4-one

2-{2-[(3-Iodobenzylamino)methyl]pent-1-ylamino]-1H-quinolin-4-one;

2-{2-[(2,3-Difluoromethylenedioxybenzylamino)methyl]pent-1-ylamino]-1H-quinolin-4-one;

2-{2-[(3-Fluoro-4-trifluoromethylbenzylamino)methyl]pent-1-ylamino]-1H-quinolin-4-one;

2-{2-[(4,5-Dibromo-2-thienylmethylamino)methyl]pent-1-ylamino]-1H-quinolin-4-one;

2-{2-[(2-Fluorenylmethylamino)methyl]pent-1-ylamino]-1H-quinolin-4-one;

2-{2-[(3-Indolylmethylamino)methyl]pent-1-ylamino]-1H-quinolin-4-one;

2-{2-[(1-Acetyl-3-indolylmethylamino)methyl]pent-1-ylamino]-1H-quinolin-4-one;

2-{2-[(2Chloro-3-trifluoromethylbenzylamino)methyl]pent-1-ylamino]-1H-quinolin-4-one;

2-{2-[(2-Chloro-5-trifluoromethylbenzylamino)methyl]pent-1-ylamino]-1H-quinolin-4-one;

2-{2-[(3,4-Dichlorobenzylamino)methyl]pent-1-ylamino]-1H-quinolin-4-one;

2-{2-[(3,5-bis-Trifluoromethylbenzylamino)methyl]pent-1-ylamino]-1H-quinolin-4-one;

2-{2-[(3,5-Dimethylbenzylamino)methyl]pent-1-ylamino]-1H-quinolin-4-one;

2-[3-(4,6-Dichloro-2-indolylmethylamino)prop-1-ylamino]-1H-quinolin-4-one;

2-[3-(4,6-Dichloro-3-methyl-2-indolylmethylamino)prop-1-ylamino]-1H-quinolin-4-one;

2-[3-(6-Methoxy-4-trifluoromethyl-2-indolylmethylamino)prop-1-ylamino]-1H-quinolin-4-one;

2-[3-(3-Trifluoromethylthiobenzylamino)prop-1-ylamino]-1H-quinolin-4-one;

2-[3-(3,4-Dichlorobenzylamino)prop-1-ylamino]-1H-quinolin-4-one;

2-[3-(3,5-Dibromobenzylamino)prop-1-ylamino]-1H-quinolin-4-one;

2-[3-(4,6-Dimethyl-2-indolylmethylamino)prop-1-ylamino]-1H-quinolin-4-one;

2-[3-(3,4-Dichlorobenzylamino)prop-1-ylamino]-6-methyl-1H-quinolin-4-one;

2-[3-(4,6-bis-Trifluoromethyl-2-indolylmethylamino)prop-1-ylamino]-1H-quinolin-4-one;

2-[3-(3-Chlorobenzylamino)prop-1-ylamino]-1H-quinolin-4-one;

2-[3-(4,5-Dibromo-2-thienylmethylamino)prop-1-ylamino]-1H-quinolin-4-one;

2-[3-(3,4-Difluorobenzylamino)prop-1-ylamino]-1H-quinolin-4-one;

2-[3-(1-Benzyl-3-indolylmethylamino)prop-1-ylamino]-1H-quinolin-4-one;

2-[3-(2-Fluoro-5-trifluoromethylbenzylamino)prop-1-ylamino]-1H-quinolin-4-one;

2-{3-[(1-Benzyl-1H-indol-3-ylmethyl)-amino]-2-methyl-propylamino}-1H-quinolin-4-one;

2-{2-[(3-Phenoxy-benzylamino)-methyl]-phenylamino}-1H-quinolin-4one;

2-{3-[(9H-Xanthen-9-ylmethyl)-amino]-propylamino}-1H-quinolin-4-one;

2-{3-[(1-Benzenesulfonyl-1H-indol-3-ylmethyl)-amino]-propylamino}-1H-quinolin-4-one;

2-(3-{[1-(1-Phenyl-methanoyl)-1H-indol-3-ylmethyl]-amino}-propylamino)-1H-quinolin-4-one;

2-{3-[(4-Bromo-5-ethyl-thiophen-2-ylmethyl)-amino]-propylamino}-1H-quinolin-4-one;

2-{3-[(6-Bromo-benzo[b]thiophen-2-ylmethyl)-amino]-propylamino}-1H-quinolin-4-one;

2-{3-[(4-Bromo-thiophen-2-ylmethyl)-amino]-propylamino}-1H-quinolin-4-one;

2-[3-(3,5-Dichloro-benzylamino)-propylamino]-1H-quinolin-4-one;

2-[3-(3-Iodo-benzylamino)-propylamino]-1H-quinolin-4-one;

2-[3-(4-Butyl-benzylamino)-propylamino]-1H-quinolin-4-one;

2-[3-(4-Chloro-3-trifluoromethyl-benzylamino)-propylamino]-1H-quinolin-4-one;

2-{2-[(2-Fluoro-4-trifluoromethyl-benzylamino)-methyl]-pentylamino}-1H-quinolin-4-one;

2-{2-[(4-Chloro-3-trifluoromethyl-benzylamino)-methyl]-pentylamino}-1H-quinolin-4-one;

2-{3-[1-(4,6-Dichloro-1H-indol-2-yl)-ethylamino]-propylamino}-1H-quinolin-4-one;

2-{3-[(4-Methoxy-6-trifluoromethyl-1H-indol-2-ylmethyl)-amino]-propylamino}-1H-quinolin-4-one;

2-{3-[(3-Cyano-1H-indol-7-ylmethyl)-amino]-propylamino}-1H-quinolin-4-one;

2-{3-[(3-Bromo-5-methyl-1H-indol-7-ylmethyl)-amino]-propylamino}-1H-quinolin-4-one;

2-{3-[(3-Bromo-5-methylsulfanyl-1H-indol-7-ylmethyl)-amino]-propylamino}-1H-quinolin-4-one;

2-{3-[(3-Chloro-5-methylsulfanyl-1H-indol-7-ylmethyl)-amino]-propylamino}-1H-quinolin-4-one;

2-{3-[(5-Chloro-3-methyl-1H-indol-7-ylmethyl)-amino]-propylamino}-1H-quinolin-4-one;

2-(2-{[(4,6-Dichloro-1H-indol-2-ylmethyl)-amino]-methyl}-pentylamino)-1H-quinolin-4-one;

2-[2-({[1-(3,5-Dibromo-benzyl)-1H-indol-3-ylmethyl]-amino}-methyl)-pentylamino]-1H-quinolin-4-one;

2-(3-{[1-(3,5-Dibromo-benzyl)-1H -indol-3-ylmethyl]-amino}-2-methyl-propylamino)-1H-quinolin-4-one;

and 2-(3-{[4,6-Dichloro-3-(pyridin-4-ylsulfanylmethyl)-1H-indol-2-ylmethyl]-amino}-propylamino)-1H-quinolin-4-one.

Most Preferred Compounds are:

2-{2-[(1-Benzyl-3-indolylmethylamino)methyl]pent-1-ylamino}-1H-quinolin-4-one;

2-[3-(4,6-Dichloro-2-indolylmethylamino)prop-1-ylamino]-1H-quinolin-4-one;

2-[3-(4-Chloro-3-trifluoromethyl-benzylamino)-propylamino]-1H-quinolin-4-one;

2-{2-[(3-Trifluoromethylthiobenzylamino)methyl]pent-1-ylamino]-1H-quinolin-4-one;

2-[2-({[1-(3,5-Dibromo-benzyl)-1H-indol-3-ylmethyl]-amino)-methyl)-pentylamino]-1H-quinolin-4-one; and

2-[3-(4-Chloro-3-trifluoromethylbenzylamino)prop-1-ylamino]-1H-quinolin-4-one.

Compounds of Formula (I) may be prepared as outlined in the following scheme:

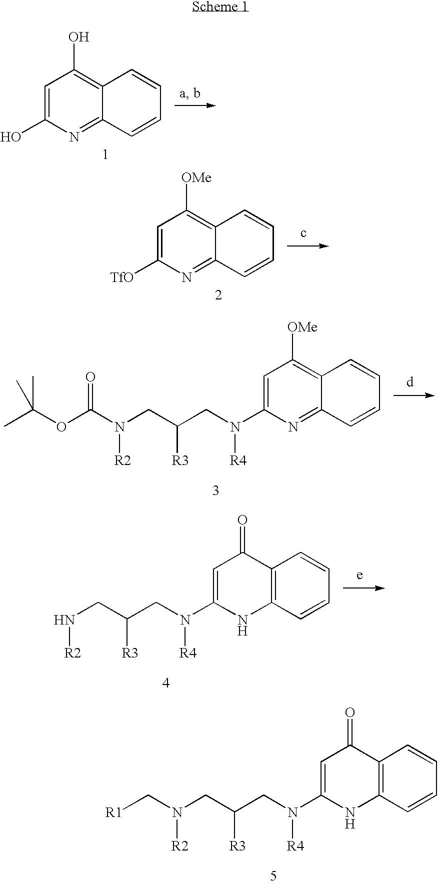

Conditions: a) Dimethylsulfate, methylene chloride, acetone, reflux; b) trifluoroacetic anhydride, pyridine, rt; c) BocR₂NCH₂CHR₃CH₂NHR₄, acetonitrile, diisopropylethyl amine, reflux; d) concentrated hydrochloric acid, reflux; e) R₁CHO, acetic acid, methanol, rt, then sodium cyanoborohydride.

Methylation of 2,4dihydroxyquinoline (1) with dimethylsulfate, followed by treatment with trifluoroacetic anhydride furnished intermediate 2, as outlined in Scheme 1 (see PCT application PCT/EP99/02648). Coupling of 2 with various mono-tert-butoxycarbonyl protected propylenediamines was accomplished in acetonitrile at reflux to give urethanes 3. Removal of the tert-butoxycarbonyl protecting group and conversion of the 4-methoxyquinoline to the corresponding quinolone with concentrated hydrochloric acid, followed by reductive alkylation of the resultant amines 4 with various aldehydes provided the target compounds 5.

In order to use a compound of the Formula (I) or a pharmaceutically acceptable salt thereof for the treatment of humans and other mammals it is normally formulated in accordance with standard pharmaceutical practice as a pharmaceutical composition.

Compounds of Formula (I) and their pharmaceutically acceptable salts may be administered in a standard manner for the treatment of the indicated diseases, for example orally, parenterally, sub-lingually, transdermally, rectally, via inhalation or via buccal administration.

Compounds of Formula (I) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, agar, pectin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils and are incorporated in a soft gelatin capsule shell.

Typical parenteral compositions consist of a solution or suspension of the compound or salt in a sterile aqueous or non-aqueous carrier optionally containing a parenterally acceptable oil, for example polyethylene glycol, polyvinylpyrrolidone, lecithin, arachis oil, or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (1) or a pharmaceutically acceptable salt thereof which is active when administered in this way, with a binding and/or lubricating agent, for example polymeric glycols, gelatins, cocoa-butter or other low melting vegetable waxes or fats or their synthetic analogues.

Typical transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer to themselves a single dose.

Each dosage unit for oral administration contains suitably from 0.1 mg to 500 mg/Kg, and preferably from 1 mg to 100 mg/Kg, and each dosage unit for parenteral administration contains suitably from 0.1 mg to 100 mg, of a compound of Formula (I) or a pharmaceutically acceptable salt thereof calculated as the free acid. Each dosage unit for intranasal administration contains suitably 1-400 mg and preferably 10 to 200 mg per person. A topical formulation contains suitably 0.01 to 1.0% of a compound of Formula (I).

The daily dosage regimen for oral administration is suitably about 0.01 mg/Kg to 40 mg/Kg, of a compound of Formula (I) or a pharmaceutically acceptable salt thereof calculated as the free acid. The daily dosage regimen for parenteral administration is suitably about 0.001 mg/Kg to 40 mg/Kg, of a compound of the Formula (I) or a pharmaceutically acceptable salt thereof calculated as the free acid. The daily dosage regimen for intranasal administration and oral inhalation is suitably about 10 to about 500 mg/person. The active ingredient may be administered from 1 to 6 times a day, sufficient to exhibit the desired activity.

These 2-(NH-substituted) quinolones may be used for the treatment of congestive heart failure, stroke, ischemic heart disease (angina, myocardial ischemia), cardiac arrhythmia, hypertension (essential and pulmonary), COPD, restenosis, asthma, neurogenic inflammation and metabolic vasculopathies, addiction, schizophrenia, impulsivity, anxiety, stress, depression, neuromuscular function, and diabetes.

No unacceptable toxicological effects are expected when compounds of the invention are administered in accordance with the present invention.

The biological activity of the compounds of Formula (I) are demonstrated by the following tests:

Radioligand Binding:

HEK-293 cell membranes containing stable cloned human and rat GPR-14 (20 ug/assay) were incubated with 200 pM [125I] h-U-II (200 Ci/mmol⁻¹ in the presence of increasing concentrations of test compounds in DMSO (0.1 nM to 10 uM), in a final incubation volume of 200 ul (20 mM Tris-HCl, 5 mM MgCl2). Incubation was done for 30 minutes at room temperature followed by filtration GF/B filters with Brandel cell harvester. ¹²⁵I labeled U-II binding was quantitated by gamma counting. Nonspecific binding was defined by ¹²⁵I U-II binding in the presence of 100 nM of unlabeled human U-II. Analysis of the data was performed by nonlinear least square fitting.

Ca²⁺-Mobilization:

A microtitre plate based Ca²⁺-mobilization FLIPR assay (Molecular Devices, Sunnyvale, Calif.) was used for the functional identification of the ligand activating HEK-293 cells expressing (stable) recombinant GPR-14. The day following transfection, cells were plated in a poly-D-lysine coated 96 well black/clear plates. After 18-24 hours the media was aspirated and Fluo 3 AM-loaded cells were exposed to various concentrations (10 nM to 30 uM) of test compounds followed by h-U-II. After initiation of the assay, fluorescence was read every second for one minute and then every 3 seconds for the following one minute. The inhibitory concentration at 50% (IC50)was calculated for various test compounds.

Inositol Phosphates Assays:

HEK-293-GPR14 cells in T150 flask were prelabeled overnight with 1 uCi myo-[³H] inositol per ml of inositol free Dulbecco's modified Eagel's medium. After labeling, the cells were washed twice with Dulbecco's phosphate-buffered saline (DPBS) and then incubated in DPBS containing 10 mM LiCl for 10 min at 37° C. The experiment was initiated by the addition of increasing concentrations of h-U-II (1 pM to 1 μM) in the absence and presence of three different concentrations (0.3, 1 and 10 uM) of test compounds and the incubation continued for an additional 5 min at 37° C. after which the reaction was terminated by the addition of 10% (final concentration) trichloroacetic acid and centrifugation. The supernatants were neutralized with 100 ul of 1M Trizma base and the inositol phosphates were separated on AG 1-X8 columns (0.8 ml packed, 100-200 mesh) in formate phase. Inositol monophosphate was eluted with 8 ml of 200 mM ammonium formate. Combined inositol di and tris phosphate was eluted with 4ml of 1M ammonium formate/0.1 M formic acid. Eluted fractions were counted in beta scintillation counter. Based on shift from the control curve K_{B} was calculated.

Activity for the compounds of this invention range from 8 nM to 1 uM.

The following examples are illustrative and are not limiting if the compounds of this invention.

### EXAMPLES

### Example 1

Preparation of 2-{3-[(1-Benzenesulfonyl-1H-indol-3-ylmethyl)-amino]-propylamino}-1H-quinolin-4-one

a) 2-Hydroxy-4-methoxyquinoline

A slurry of 2,4-dihydroxyquinoline (20.7 g, 0.13 mol), potassium carbonate (35.5 g, 0.26 mol), and dimethyl sulfate (14.6 ml, 0.15 mol) in acetone (800 ml) was heated at reflux for 3 days. The reaction was cooled to ambient temperature then evaporated under reduced pressure. The residue was slurried in a system of water (1000 ml) and ethyl acetate (500 ml) for 1 hour. The solids were collected then rinsed with water (3×250 ml) and ethyl ether (3×250 ml). Vacuum dried over phosphorus pentoxide to give 2-hydroxy-4-methoxyquinoline (16.3 g, 72%) as tan powder. M+CH3CN=217.

b) 1,1,1-Trifluoromethanesulfonic acid 4-methoxyquinolin-2-yl ester

A slurry of 2-hydroxy-4-methoxyquinoline (13.4 g, 76.6 mmol) in pyridine (75 ml) was slowly treated under argon with trifluoromethanesulfonic ahydride (15.5 ml, 91.2 mmol). The reaction was allowed to stir at ambient temperature. After 4 days, the reaction was evaporated under reduced pressure to an oil that was dried by azeotrope with toluene (2×200 ml) to give the crude product as a brown solid. Flash chromatography on silica (1:1 ethyl acetate/hexanes as eluent) gave 1,1,1-trifluoromethanesulfonic acid 4-methoxyquinolin-2-yl ester (20.4 g, 87%) as a yellow oil that solidified on standing. [M+H]⁺ 308, M+CH3CN=349.

c) [3-(4-Methoxyquinolin-2-ylamino)propyl]carbamic acid tert-butyl ester

A solution of 1,1,1-trifluoromethanesulfonic acid 4-methoxyquinolin-2-yl ester (4.51 g, 14.7 mmol), tert-butyl N-(3-aminopropyl)carbamate (3.07 g, 17.6 mmol), and diisopropylethylamine (3.84 ml, 22.0 mmol) in anhydrous acetonitrile (35 ml) was heated at reflux for 6 days. Cooled to ambient temperature then evaporated under reduced pressure to an oil. Taken into water (35 ml) then extracted into ethyl acetate. The extracts were dried (sodium sulfate) then concentrated to an oil. Column chromatography on silica (1:1 ethyl acetate/hexanes) gave [3-(4-methoxyquinolin-2-ylamino)propyl]carbamic acid tert-butyl ester (3.10 g, 64%) as a colorless oil that solidified on standing. [M+H]⁺ 332.

d) 3-(Aminoprop-1-ylamino)-1H-quinolin-4-one dihyrochloride

A solution of [3-(4-methoxyquinolin-2-ylamino)propyl]carbamic acid tert-butyl ester (650 mg, 2.0 mmol) in concentrated hydrochloric acid (35 ml) was heated at reflux for 24 hours, at which time it was allowed to cool to room temperature and evaporated under reduced pressure. The resultant oil was diluted with toluene and evaporated under reduced pressure to give 3-(aminoprop-1-ylamino)-1H-quinolin-4-one dihydrochloride (570 mg, 100%) as a white solid. [M+H]⁺ 218.

e) 2-{3-[(1-Benzenesulfonyl-1H-indol-3-ylmethyl)-amino]-propylamino}-1H-quinolin-4-one

A solution of 3-(aminoprop-1-ylamino)-1H-quinolin-4-one dihydrochloride (240 mg, 0.83 mmol) and 1-benzenesulfonyl-3-indole carboxaldehyde (240 mg, 0.83 mmol) in methanol (40 ml) was treated with glacial acetic acid (20 drops) and sodium methoxide (95%, 89 mg, 1.7 mmol). The reaction stirred at ambient temperature for 24 hours then was treated with a solution of sodium cyanoborohydride (100 mg, 1.7 mmol) in methanol (2.0 ml). The reaction stirred at ambient temperature for 24 hours, at which time it was evaporated under reduced pressure. The resultant residue was diluted with saturated aqueous sodium chloride (30 ml) and 10% aqueous sodium hydroxide (30 ml) and extracted with ethyl acetate (2×30 mL). The extracts ware dried (sodium sulfate) and concentrated. Purification by reverse phase HPLC (water/acetonitrile, 9:1 to 1:9 gradient) gave 2-{3-[(1-Benzenesulfonyl-1H-indol-3-ylmethyl)-amino]-propylamino}-1H-quinolin-4-one (200 mg, 50%) as a white solid. [M+H]⁺ 487.

### Examples 2-9

| | | MS (ES+) m/e |
|---|---|---|
| Example | Compound | [M + H] |
| 2 | 2-{3-[(1H-indol-3-ylmethyl)-amino]-2-methyl- | 451 |
| | propylamino}-1H-quinolin-4-one | |
| 3 | 2-{2-[(3-Phenoxy-benzylamino)-methyl]-pentylamino}-1H- | 442 |
| | quinolin-4-one | |
| 4 | 2-(3-{[1-(3,5-Dibromo-benzyl)-1H-indol-3-ylmethyl]- | |
| | amino}-2-methyl-propylamino)-1H-quinolin-4-one | 609 |
| 5 | 2-(2-{[(4,6-Dichloro-1H-indol-2-ylmethyl)-amino]-methyl}- | 457 |
| | pentylamino)-1H-quinolin-4-one | |
| 6 | 2-[2-({[1-(3,5-Dibromo-benzyl)-1H-indol-3-ylmethyl]- | 635 |
| | amino}-methyl)-pentylamino]-1H-quinolin-4-one | |
| 7 | 2-{3-[(9H-Xanthen-9-ylmethyl)-amino]-propylamino}-1H- | 412 |
| | quinolin-4-one | |
| 8 | 2-(3-{[1-(1-Phenyl-methanoyl)-1H-indol-3-ylmethyl]- | 451 |
| | amino}-propylamino)-1H-quinolin-4-one | |

### Example 9

Formulations for pharmaceutical use incorporating compounds of the present invention can be prepared in various forms and with numerous excipients. Examples of such formulations are given below.

Inhalant Formulation

A compound of Formula I, (1 mg to 100 mg) is aerosolized from a metered dose inhaler to deliver the desired amount of drug per use.

| Tablets/Ingredients | | Per Tablet |
|---|---|---|
| 1. | Active ingredient | 40 mg |
| | (Cpd of Form. I) | |
| 2. | Corn Starch | 20 mg |
| 3. | Alginic acid | 20 mg |
| 4. | Sodium Alginate | 20 mg |
| 5. | Mg stearate | 1.3 mg |
| | | 2.3 mg |

Procedure for Tablets:

Step 1 Blend ingredients No. 1, No. 2, No. 3 and No. 4 in a suitable mixer/blender.

Step 2 Add sufficient water portion-wise to the blend from Step 1 with careful mixing after each addition. Such additions of water and mixing until the mass is of a consistency to permit its conversion to wet granules.

Step 3 The wet mass is converted to granules by passing it through an oscillating granulator using a No. 8 mesh (2.38 mm) screen.

Step 4 The wet granules are then dried in an oven at 140° F. (60° C.) until dry.

Step 5 The dry granules are lubricated with ingredient No. 5.

Step 6 The lubricated granules are compressed on a suitable tablet press.

Parenteral Formulation

A pharmaceutical composition for parenteral administration is prepared by dissolving an appropriate amount of a compound of formula I in polyethylene glycol with heating. This solution is then diluted with water for injections Ph Eur. (to 100 ml). The solution is then sterilized by filtration through a 0.22 micron membrane filter and sealed in sterile containers.

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

What is claimed is:

1. A method of treating conditions associated with Urotensin-II imbalance by antagonizing the Urotensin-II receptor which comprises administering to a patient in need thereof, a compound of Formula I: 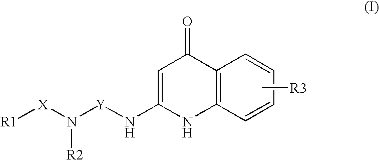
wherein:
R₁ is phenyl, thienyl, benzothienyl, benzhydryl, xanthenyl, napthyl, or indolyl, all of which may be substituted or unsubstituted by one or two substituients selected from: halogens, —CN, CH₃CO—, (C₁₋₆)alkyl, mono to perfluoro(C₁₋₃)alkyl, (C₂₋₆)alkenyl, (C₁₋₆)alkoxy, (C₅₋₁₀)aryloxy, phenylC₍₁₋₆₎alkoxy, —OH, —NH₂, mono- or di-(C₁₋₆)alkylamino, —NO₂, —CO₂H, —CO₂(C₁₋₆)alkyl, —S(C₁₋₆)alkyl, —SO₂(C₁₋₆)alkyl, H₂NSO₂—, —CONH₂, —SO₂(C₅₋₁₀)aryl, or —CO₂N{(C₁₋₆)alkyl}2; (add benzyl wording)
R₂ is hydrogen or Me;
R₃ is hydrogen, I, F, Br, Cl, C₁₋₆alkyl, C₁₋₆alkoxy, —OH, or —CN;
X is —CH(R₄)— or CO;
R₄ is hydrogen, CO, C₁₋₆ alkyl, or phenyl;
Y is —CH₂C(R₅)(R₆)CH₂—;
R₅ and R₆ are independently hydrogen or C₁₋₆ alkyl.

2. A method according to claim 1 wherein R₁ is substituted phenyl, thienyl, benzothienyl, benzhydryl, or indolyl; R₂ is hydrogen; R₃ is hydrogen, halo, or alkyl; X is CH₂; and Y is CH₂CR₅R₆CH₂, where R₅, and R₆ are hydrogen, or R₅ is hydrogen; and R₆ is n-propyl or methyl.

3. A method according to claim 2 wherein R₁ is 1-benzyl-3-indolyl, 4,6-dichloro-2-indolyl, 3-trifluoromethylthiophenyl, 2-fluoro-5-trifluoromethylphenyl, 4,6-dichloro-3-methyl-2-indolyl, 6-methoxy-4-trifluoromethyl-2-indolyl, 3,4-dichlorophenyl, 3,5-dibromophenyl, 4chloro-3-trifluoromethylbenzyl or 3,5-Dibromobenzyl)-3-indolyl; R₂ is hydrogen; R₃ is hydrogen, Cl, or Me; X is CH₂; and Y is CH₂CR₅R₆CH₂, wherein R₅, and R₆ are H; or R₅ is hydrogen and R₆ is n-propyl, or methyl.

4. A method according to claim 1 wherein the compound is selected from:
2-{2-[(1-Benzyl-3-indolylmethylamino)methyl]pent-1-ylamino]-1H-quinolin-4-one;
2-{2-[(3-Trifluoromethylthiobenzylamino)methyl]pent-1-ylamino]-1H-quinolin-4-one;
2-{2-[(2-Fluoro-5-trifluoromethylbenzylamino)methyl]pent-1-ylamino]-1H-quinolin-4-one;
2-{2-[(4-n-Butylbenzylamino)methyl]pent-1-ylamino]-1H-quinolin-4-one;
2-{2-[(2,5-Dimethyl-1-phenyl-3-pyrrolylmethylamino)methyl]pent-1-ylamino]-1H-quinolin-4-one;
2-{2-[(1-Bromo-2-naphthylmethylamino)methyl]pent-1-ylamino]-1H-quinolin-4-one;
2-{2-[(4-Bromo-3,5-dimethoxybenzylamino)methyl]pent-1-ylamino]-1H-quinolin-4-one;
2-{2-[(5-Bromo-2-fluorobenzylamino)methyl]pent-1-ylamino]-1H-quinolin-4-one
2-{2-[(3-Iodobenzylamino)methyl]pent-1-ylamino]-1H-quinolin-4-one;
2-{2-[(2,3-Difluoromethylenedioxybenzylamino)methyl]pent-1-ylamino]-1H-quinolin-4-one;
2-{2-[(3-Fluoro-4-trifluoromethylbenzylamino)methyl]pent-1-ylamino]-1H-quinolin-4-one;
2-{2-[(4,5-Dibromo-2-thienylmethylamino)methyl]pent-1-ylamino]-1H-quinolin-4-one;
2-{2-[(2-Fluorenylmethylamino)methyl]pent-1-ylamino]-1H-quinolin-4-one;
2-{2-[(3-Indolylmethylamino)methyl]pent-1-ylamino]-1H-quinolin-4-one;
2-{2-[(1-Acetyl-3-indolylmethylamino)methyl]pent-1-ylamino]-1H-quinolin-4-one;
2-{2-[(2-Chloro-3-trifluoromethylbenzylamino)methyl]pent-1-ylamino]-1H-quinolin-4-one;
2-{2-[(2-Chloro-5-trifluoromethylbenzylamino)methyl]pent-1-ylamino]-1H-quinolin-4-one;
2-{2-[(3,4-Dichlorobenzylamino)methyl]pent-1-ylamino]-1H-quinolin-4-one;
2-{2-[(3,5-bis-Trifluoromethylbenzylamino)methyl]pent-1-ylamino]-1H-quinolin-4-one;
2-{2-[(3,5-Dimethylbenzylamino)methyl]pent-1-ylamino]-1H-quinolin-4-one;
2-[3-(4,6-Dichloro-2-indolylmethylamino)prop-1-ylamino]-1H-quinolin-4-one;
2-[3-(4,6-Dichloro-3-methyl-2-indolylmethylamino)prop-1-ylamino]-1H-quinolin-4-one;
2-[3-(6-Methoxy-4-trifluoromethyl-2-indolylmethylamino)prop-1-ylamino]-1H-quinolin-4-one;
2-[3-(3-Trifluoromethylthiobenzylamino)prop-1-ylamino]-1H-quinolin-4-one;
2-[3-(3,4-Dichlorobenzylamino)prop-1-ylamino]-1H-quinolin-4-one;
2-[3-(3,5-Dibromobenzylamino)prop-1-ylamino]-1H-quinolin-4-one;
2-[3-(4,6-Dimethyl-2-indolylmethylamino)prop-1-ylamino]-1H-quinolin-4-one;
2-[3-(3,4-Dichlorobenzylamino)prop-1-ylamino]-6-methyl-1H-quinolin-4-one;
2-[3-(4,6-bis-Trifluoromethyl-2-indolylmethylamino)prop-1-ylamino]-1H-quinolin-4-one;
2-[3-(3-Chlorobenzylamino)prop-1-ylamino]-1H-quinolin-4-one;
2-[3-(4,5-Dibromo-2-thienylmethylamino)prop-1-ylamino]-1H-quinolin-4-one;
2-[3-(3,4-Difluorobenzylamino)prop-1-ylamino]-1H-quinolin-4-one;
2-[3-(1-Benzyl-3-indolylmethylamino)prop-1-ylamino]-1H-quinolin-4-one;
2-[3-(2-Fluoro-5-trifluoromethylbenzylamino)prop-1-ylamino]-1H-quinolin-4-one;
2-{3-[(1-Benzyl-1H-indol-3-ylmethyl)-amino]-2-methyl-propylamino}-1H-quinolin-4-one;
2-{2-[(3-Phenoxy-benzylamino)-methyl]-pentylamino}-1H-quinolin-4-one;
2-{3-[(9H-Xanthen-9-ylmethyl)-amino]-propylamino}-1H-quinolin-4-one;
2-{3-[(1-Benzenesulfonyl-1H-indol-3-ylmethyl)-amino]-propylamino}-1H-quinolin-4-one;
2-(3-{[1-(1-Phenyl-methanoyl)-1H-indol-3-ylmethyl]-amino}-propylamino)-1H-quinolin-4-one;
2-{3-[(4-Bromo-5-ethyl-thiophen-2-ylmethyl)-amino]-propylamino}-1H-quinolin-4-one;
2-{3-[(6-Bromo-benzo[b]thiophen-2-ylmethyl)-amino]-propylamino}-1H-quinolin-4-one;
2-{3-[(4-Bromo-thiophen-2-ylmethyl)-amino]-propylamino}-1H-quinolin-4-one;
2-[3-(3,5-Dichloro-benzylamino)-propylamino]-1H-quinolin-4-one;
2-[3-(3-Iodo-benzylamino)-propylamino]-1H-quinolin-4-one;
2-[3-(4-Butyl-benzylamino)-propylamino]-1H-quinolin-4-one;
2-[3-(4-Chloro-3-trifluoromethyl-benzylamino)-propylamino]-1H-quinolin-4-one;
2-{2-[(2-Fluoro-4-trifluoromethyl-benzylamino)-methyl]-pentylamino}-1H-quinolin-4-one;
2-{2-[(4-Chloro-3-trifluoromethyl-benzylamino)-methyl]-pentylamino}-1H-quinolin-4-one;
2-{3-[1-(4,6-Dichloro-1H-indol-2-yl)-ethylamino]-propylamino}-1H-quinolin-4-one;
2-{3-[(4-Methoxy-6-trifluoromethyl-1H-indol-2-ylmethyl)-amino]-propylamino}-1H-quinolin-4-one;
2-{3-[(3-Cyano-1H-indol-7-ylmethyl)-amino]-propylamino}-1H-quinolin-4-one;
2-{3-[(3-Bromo-5-methyl-1H-indol-7-ylmethyl)-amino]-propylamino}-1H-quinolin-4-one;
2-{3-[(3-Bromo-5-methylsulfanyl-1H-indol-7-ylmethyl)-amino]-propylamino}-1H-quinolin-4-one;
2-{3-[(3-Chloro-5-methylsulfanyl-1H-indol-7-ylmethyl)-amino]-propylamino}-1H-quinolin-4-one;
2-{3-[(5-Chloro-3-methyl-1H-indol-7-ylmethyl)-amino]-propylamino}-1H-quinolin-4-one;
2-(2-{[(4,6-Dichloro-1H-indol-2-ylmethyl)-amino]-methyl}-pentylamino)-1H-quinolin-4-one;
2-[2-({[1-(3,5-Dibromo-benzyl)-1H-indol-3-ylmethyl]-amino}-methyl)-pentylamino]-1H-quinolin-4-one;
2-(3-{[1-(3,5-Dibromo-benzyl)-1H-indol-3-ylmethyl]-amino}-2-methyl)-propylamino]-1H-quinolin-4-one;
and 2-(3-{[4,6-Dichloro-3-(pyridin-4-ylsulfanylmethyl)-1H-indol-2-ylmethyl]-amino}-propylamino)-1H-quinolin4-one.

5. A method according to claim 1 wherein the compound is selected from:
2-{2-[(1-Benzyl-3-indolylmethylamino)methyl]pent-1-ylamino}-1H-quinolin-4-one;
2-[3-(4,6-Dichloro-2-indolylmethylamino)prop-1-ylamino]-1H-quinolin-4-one;
2-[3-(4-Chloro-3-trifluoromethyl-benzylamino)-propylamino]-1H-quinolin-4-one;
2-{2-[(3-Trifluoromethylthiobenzylamino)methyl]pent-1-ylamino]-1H-quinolin-4-one;
2-[2-({[1-(3,5-Dibromo-benzyl)-1H-indol-3-ylmethyl]-amino}-methyl)-pentylamino]-1H-quinolin-4-one; and
2-[3-(4-Chloro-3-trifluoromethylbenzylamino)prop-1-ylamino]-1H-quinolin-4-one.

6. A method according to claim 1 wherein the disease is congestive heart failure, stroke, ischemic heart disease, angina, myocardial ischemia, cardiac arrythmias, essential hypertension, pulmonary hypertension, COPD, restenosis, asthma, neurogenic inflammation metabolic vasculopathies, addiction, schizophrenia, impulsivity, anxiety, stress, depression, neuromuscular function, or diabetes.
